(19) United States
(12) Patent Application Publication
(10) Pub. No.: US 2019/0228545 A1
(43) Pub. Date: Jul. 25, 2019

(54) SYSTEM AND METHOD FOR PARALLELIZATION OF CPU AND GPU PROCESSING FOR ULTRASOUND IMAGING DEVICES

(71) Applicant: COVIDIEN LP, MANSFIELD, MA (US)

(72) Inventors: Xiaofang Gan, Shanghai (CN); Wei Tan, Shanghai (CN)

(21) Appl. No.: 16/338,518

(22) PCT Filed: Sep. 28, 2016
(86) PCT No.: PCT/CN2016/100528

§ 371 Date: Apr. 1, 2019

(51) Int. Cl. G06T 11/00; A61B 8/14; A61B 8/00; G06T 1/20

(52) U.S. Cl. CPC G06T 11/003; A61B 8/145; G06T 1/20; A61B 8/4488; A61B 8/461; A61B 8/4483

(57) ABSTRACT

An ultrasound imaging system includes a transducer array, an ultrasound frontend, and a processing apparatus. The transducer array has a plurality of transducer elements, each of the plurality of transducer elements configured to transmit acoustic energy to a region of interest and receive reflected acoustic energy. The ultrasound frontend digitally samples the reflected acoustic in energy to genera the radio frequency (RF) data. The processing apparatus includes a central processing unit (CPU), a first in/first out (FIFO) buffer, and a graphical processing unit (GPU). The CPU receives the RF data including RF frames and the FIFO buffer includes a plurality of memory blocks for storing the RF frames, wherein a size of each memory block is equal to the size of a single RF frame. The GPU reads the RF frames from the plurality of memory blocks of the FIFO buffer and reconstructs an image.

### BACKGROUND

### 1. Technical Field

The present disclosure relates generally to ultrasound imaging devices, and, more particularly, to a system and method for parallelization of CPU and GPU processing of ultrasound imaging devices.

### 2. Discussion of Related Art

An ultrasound system has become a popular diagnostic tool since it has a wide range of applications. Specifically, due to its non-invasive and non-destructive nature, the ultrasound system has been extensively used in the medical profession. Modern high-performance ultrasound systems and techniques are commonly used to produce two or three-dimensional images of internal features of an object (e.g., human organs).

The ultrasound system generally uses a probe containing a wide bandwidth transducer to transmit and receive ultrasound signals. The ultrasound system forms images of human internal tissues by electrically exciting an acoustic transducer element or an array of acoustic transducer elements to generate ultrasound signals that travel into the body. The ultrasound signals produce ultrasound echo signals since they are reflected from body tissues, which appear as discontinuities to the propagating ultrasound signals. Various ultrasound echo signals return to the transducer element and are converted into electrical signals, which are amplified and processed to produce ultrasound data for an image of the tissues.

The ultrasound system employs an ultrasound probe containing a transducer array for transmission and reception of ultrasound signals. The ultrasound signals are transmitted along scan lines aligned with the direction of a scan head of the ultrasound probe. The ultrasound system forms ultrasound images based on the received ultrasound signals. The technique of transmitting the ultrasound signals by steering the scan lines has been used to obtain an ultrasound image having a wider view angle.

Moreover, an ultrasound imaging system may include an ultrasound diagnostic unit and an image processing unit. The ultrasound diagnostic unit may transmit ultrasound signals to a target object and form, for example, 12-bit data based on echo signals. The image processing unit may form an ultrasound image based on the 12-bit data. The image processing unit may also include a digital signal processing unit (DSP), a digital scan converter (DSC) and a central processing unit (CPU). The DSP may be operable to process the 12-bit data to form 12-bit raw data for forming a brightness (B) mode image, an M mode image, or a color Doppler mode image. The DSC may be operable to scan-convert the raw data to thereby output scan-converted data suitable for a display format. The CPU may be operable to control operations of the DSP, DSC, and a display unit. Also, the CPU may be further operable to perform filtering and rendering upon the scan-converted data to thereby form pixel data for image modes.

The rendering and formation of the pixel data performed in the CPU may require a large amount of data operations so that fewer CPU resources are available for other processes and power consumption by the CPU becomes higher. In addition, the CPU has to control data input/output at the DSP and DSC. Thus, an excessive load may be applied to the CPU in forming the ultrasound image so that the CPU is not available to provide a higher frame rate of ultrasound images. Accordingly, there is a need for systems and methods for relieving loads from the CPU, and providing a higher frame rate of ultrasound images.

### SUMMARY

In one aspect, the present disclosure is directed to an ultrasound imaging system including a transducer array, an ultrasound frontend, and a processing apparatus. The transducer array has a plurality of transducer elements, each of the plurality of transducer elements configured to transmit acoustic energy to a region of interest and receive reflected acoustic energy. The ultrasound frontend samples the reflected acoustic energy to generate radio frequency (RF) data. The processing apparatus includes a central processing unit (CPU), a first in/first out (FIFO) buffer, and a graphical processing unit (GPU). The CPU receives the RF data including RF frames and the FIFO buffer includes a plurality of memory blocks for storing the RF frames, wherein a size of each memory block is equal to the size of a single RF frame. The GPU reads the RF frames from the plurality of memory blocks of the FIFO buffer and reconstructs an image.

In the disclosed embodiments, the ultrasound imaging system further comprises a display for displaying a reconstructed image of the region of interest.

In the disclosed embodiments, the image is reconstructed by performing envelope detection, compounding, and post-processing.

In the disclosed embodiments, the number of the plurality of memory blocks of the FIFO buffer is greater than or equal to (t2+t3)/t1, where t1 is the time that the CPU receives one RF frame, t2 is the time that the GPU reads one RF frame, and t3 is the time that the GPU performs envelope detection, compounding, and post-processing.

In the disclosed embodiments, the CPU receives the RF frames and the GPU reads the RF frames, in a parallel manner.

In the disclosed embodiments, the number of the plurality of transducer elements is 128.

In the disclosed embodiments, the acoustic energy is transmitted in plane waveform, which has a plurality of steering angles. The number of steering angles is 11.

In the disclosed embodiments, the GPU reads a single memory block of the FIFO buffer to process one RF frame.

In the disclosed embodiments, the GPU performs beamforming processing by delay-and-sum operations in a parallel manner.

In one aspect, the present disclosure is directed to an ultrasonic imaging method. The method includes transmitting acoustic energy to a region of interest by a transducer array including a plurality of transducer elements, receiving reflected acoustic energy, digitally sampling the reflected acoustic energy to generate RF data, receiving the RF data including RF frames by a central processing unit (CPU), storing a RF frame in a memory block of a plurality of memory blocks of a first in/first out (FIFO) buffer; reading the RF frame by a graphics processing unit (GPU) from the memory block of the FIFO buffer, and reconstructing an image based on the RF frame by the GPU. The size of each memory block is equal to the size of a single RF data.

In the disclosed embodiments, the method further includes displaying the reconstructed image of the region of interest on a display.

In the disclosed embodiments, reconstructing the image includes performing envelope detection, compounding, and post-processing by the GPU.

In the disclosed embodiments, the size of each memory block of the FIFO buffer is greater than or equal to (t2+t3)/t1, where t1 is the time that the CPU receives one RF frame, t2 is the time that the GPU reads the RF frame from the memory block, and t3 is the time that the GPU reconstructs the image.

In the disclosed embodiments, receiving the RF data and reading the RF frame are performed in a parallel manner.

In the disclosed embodiments, the number of plurality of transducer elements is 128.

In the disclosed embodiments, the acoustic energy is transmitted in plane waveform, which includes steering angles. The number of steering angles is 11.

In the disclosed embodiment, the method further includes performing beamforming process on the RF frame by the GPU.

In the disclosed embodiments, the beamforming processing is performed by delay-and-sum operation in a parallel manner.

In the disclosed embodiments, the method further includes performing beamforming process on the RF frame by the GPU.

Further, to the extent consistent, any of the aspects described herein may be used in conjunction with any or all of the other aspects described herein.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

Various aspects of the present disclosure are described hereinbelow with reference to the drawings, which are incorporated in and constitute a part of this specification, wherein:

FIG. 1 illustrates a top level architecture of an ultrasound imaging system, in accordance with aspects of the present disclosure;

FIG. 2 illustrates an image reconstruction procedure of a conventional ultrasound imaging system;

FIG. 3 illustrates an image reconstruction procedure, in accordance with aspects of the present disclosure;

FIGS. 4A and 4B illustrate CPU (central processing unit) and GPU (graphics processing unit) working flow, where FIG. 4A is the working flow when L_FIFO<(t2+t3)/t1, and FIG. 413 is the working flow when L_FIFO>(t2+t3)/t1, in accordance with aspects of the present disclosure;

FIG. 5 illustrates a block diagram showing receiving beamforming with time delays, in accordance with aspects of the present disclosure; and

FIG. 6 illustrates data-level parallelism in delay-and-sum operations, in accordance with aspects of the present disclosure.

### DETAILED DESCRIPTION

A detailed description is provided with reference to the accompanying drawings. One of ordinary skill in the art will realize that the following description is illustrative only and is not in any way limiting. Other embodiments of the present disclosure will readily suggest themselves to such skilled persons having the benefit of this disclosure.

As discussed in further detail below, various embodiments of transducer elements of an ultrasound probe communicatively coupled to an imaging system are provided with respect to waveform generation proximate to the transducer elements of the ultrasound probe. In one embodiment, the ultrasound probe is electronic, reusable, capable of precise waveform timing and intricate waveform shaping for a plurality of independent transducer elements, and capable of communicating analog or digitized data to the imaging system.

The present disclosure describes a method for increasing frame rates of ultrasound systems by parallelizing CPU and GPU. First, the CPU receives RF data from the ultrasound frontend via a USB 3.0 port, and then stores the RF data in the First In/First Out buffers (FIFO). Second, the GPU reads RF data from a FIFO buffer and then performs beamforming, envelope detection, compounding, and post processing. Third, the reconstructed image is displayed on one or more display screens. By migrating beamforming from the ultrasound frontend to the GPU, the cost of the ultrasound system may be reduced. Further, by parallelizing the receiving of RF data and beamforming, the frame rate is also further increased.

FIG. 1 illustrates an ultrasound imaging system 100, in accordance with aspects of the present disclosure. Ultrasound imaging is a non-invasive subsurface imaging modality widely used in diagnosis, screening, and as an intra-operative surgical guide. FIG. 1 depicts a transducer 110, an ultrasound frontend 120, a universal serial bus (USB) port 130, a computing device 140, and a display 150.

The transducer 110 includes a plurality of transducer elements, which are typically formed of a piezoelectric material and referred to as a transducer array. Scan lines or channels correspond to each transducer element of the transducer array. When electric signals having a frequency in the radio frequency (RF) range are provided to each transducer element of the transducer 10, each transducer element is energized to generate acoustic signals.

When the plurality of transducer elements of the transducer generate an ultrasound waveform and transmit them towards a target, the plurality of transducer elements of the transducer use time delays based on distance differences between each transducer element and the target so that each generated ultrasound waveform can reach the target at the same time.

The ultrasound waveforms are transmitted along scan lines or channels aligned with the direction of a scan head of an ultrasound probe. The ultrasound waveform is reflected by the target. The reflected waveforms can be detected by the corresponding transducer elements of the transducer 110, which in turn generates electric signals. Since the temporal shape of the reflected signals or echoes is similar to a temporal shape of RF data, the generated electrical signals based on the echoes are called RF data.

In an aspect, the transducer array 110 may include a multi-element linear, curved linear, phased linear, sector, or wide view array. For example, the transducer array 110 may provide for 16, 32, 64 or 128 channels. In one embodiment, the transducer array 110 includes 128 channels.

The received signals, echoes of the transmitted acoustic signal are converted by the transducer to RF data and then transmitted to the ultrasound frontend 120. The transducer array 110 may be incorporated into the ultrasound frontend 120. The ultrasound frontend 120 may include a signal receiver and an analog-to-digital converter (ADC). The signal receiver may perform, for example, low-noise amplification, programmable gain amplification, and low-pass filtering, and the ADC digitally samples the RF data. According to an aspect of the present disclosure, beam forming is not performed by the ultrasound frontend 120 but by the computing device 140. Beamforming is a process which combines RF data received from the plurality of transducer elements of the transducer 110 to a single signal which is focused at a specific spatial location in the space of interest. Thus, the computing device 140 does not have to wait until the ultrasound frontend 120 finishes beamforming. In this way, the total processing time can be decreased

In an aspect, the transducer array may include 128 transducer elements, which corresponds to 128 lines or channels. As a result, a single frame (image) includes 128 lines of RF data. During analog-to-digital conversion, every single line is sampled as 4096 points. Every point occupies 2 bytes, which means that the size of one frame (hereinafter a RF frame) of RF data is: 4096*128*2=1 M bytes. After performing analog-to-digital conversion, the digital RF data is transmitted to the computing device 140 via the USB 3.0 port 130. The normal transmission speed of the USB 3.0 port 130 is 300 MB/s, which means that 3.33 ms are needed to transmit one RF frame of RF data from the ultrasound frontend 120 to the computing device 140.

The computing device 140 performs beamforming and post processing. Post processing of the single beam formed signal results in the construction of ultrasound images. The images is transmitted to and displayed on a screen of the monitor 150.

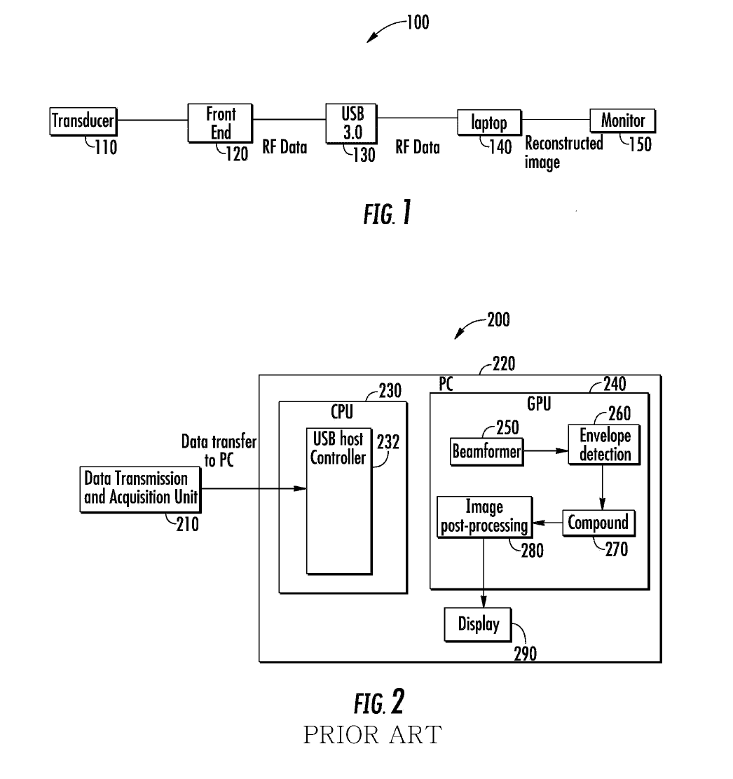

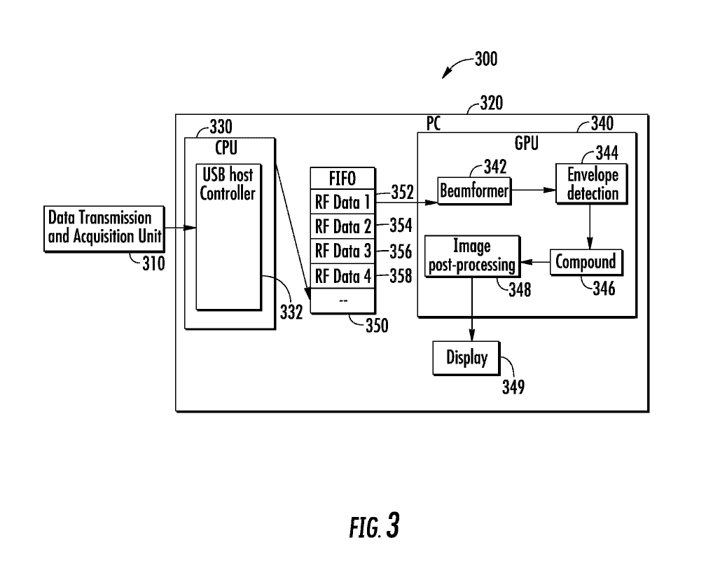

FIG. 2 illustrates an image reconstruction procedure of a conventional ultrasound imaging system of FIG. 1. The image reconstruction system 200 includes a data transmission and acquisition unit 210 and a computing device 220. The data transmission and acquisition unit 210 transfers RF data to the computing device 220. The computing device 220 may be a personal computer. The computing device 220 includes a CPU 230, a GPU 240, and a display 290. The CPU 230 is capable of functioning as at least a USB host controller 232. The GPU 240 is capable of functioning as a beamformer 250, an envelope detection unit 260, a compounding unit 270, and an image post-processing unit 280. The CPU 230 controls the USB host controller 232 to receive the RF data from the data transmission and acquisition unit 210 via a transmission port, for example a USB 3.0 port. The RF data is processed by the GPU 240, as the data is received from the CPU 230. Since the CPU 230 and the GPU 240 serially process the RF data, the CPU 230 cannot provide received RF data to the GPU 240 until the GPU 240 finishes processing RF data, and the GPU 240 has to wait until the CPU 230 finishes reception of RF data. Thus, serial processes between the CPU 230 and the GPU 240 cause unnecessary waiting times in the CPU 230 and/or the GPU 240.

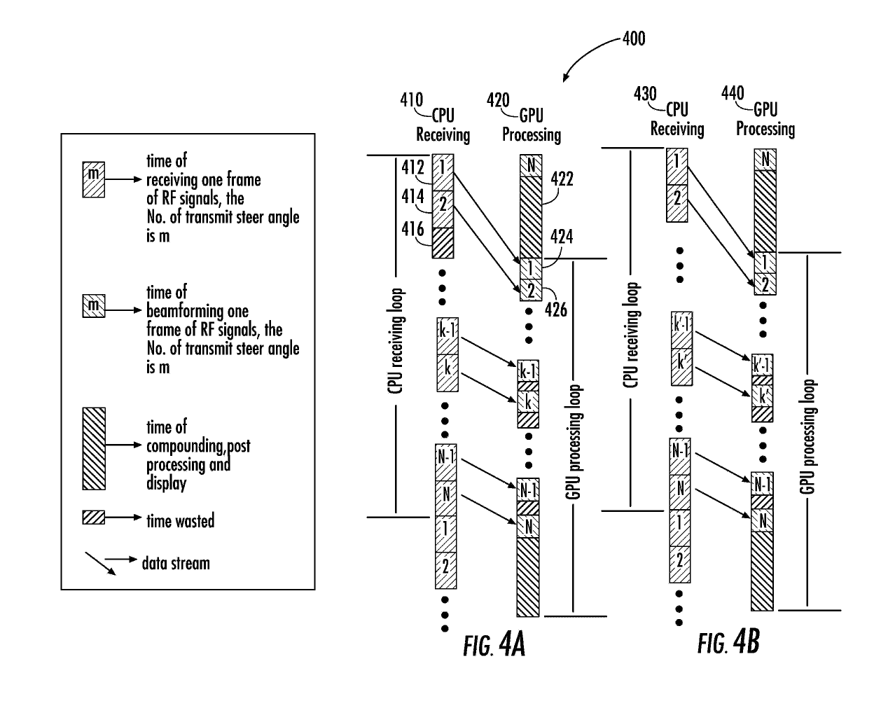

FIG. 3 illustrates an image reconstruction system in accordance with aspects of the present disclosure.

The image reconstruction system 300 includes a data transmission and acquisition unit 310, a computing device 320, and a display 349. The data transmission and acquisition unit 310 transfers data to the computing device 320. The computing device 320 may be a personal computer, a tablet, or a smart device (e.g., a smartphone). The computing device 320 includes a CPU 330, a GPU 340, and a FIFO buffer 350. The CPU 330 may include at least a USB host controller 332 so as to control a data transfer port (e.g., the USB 3.0 port 130 of FIG. 1) to receive RF data from the data transmission and acquisition unit 310. When the RF data is received, the CPU 330 processes the RF data to form RF frames.

The FIFO buffer 350 is coupled with the CPU 330 and the GPU 340. The CPU 330 stores each RF frame in the FIFO buffer 350 when the FIFO buffer 350 has unoccupied spaces, and then receives RF frames. The FIFO buffer 350 may include a plurality of memory blocks 352, 354, 356, 358, etc. One skilled in the art may contemplate any size FIFO buffer having thousands of memory blocks for storing data. The size of one memory block (e.g., 352) of the FIFO buffer 350 may be equal to the size of one RF frame of the RF data. Thus, each RF frame is stored in one memory block (e.g., 352) of the FIFO buffer 350.

The GPU 340 may include a beamformer 342, an envelope detection unit 344, a compounding unit 346, and an image post-processing unit 348. When the GPU 340 reads a RF frame from one memory block (e.g., 352) of the FIFO buffer 350, the beamformer 342 processes the RF frame by delaying and summing digital data to generate a single signal which is focused at a specific location in an image. The envelope detection unit 344 detects envelope of the signals generated by the beamformer 342, thus removing the carrier signal. Since the image generated from the envelop detection unit 344 includes speckle errors (e.g., coherent noise), which result from constructive and destructive wave interference of reflections of the ultrasound waves generated by the plurality of transducer elements of the transducer 110 of FIG. 1.

The compounding unit 346 removes the speckle errors. For example, the compounding unit 346 may remove the speckle errors by averaging pixel values located at the same location of multiple images obtained by using different steering angles. The compounding unit 346 may perform removal of the speckle errors by any means readily available to a person having ordinary skill in the art. After the compounding process, an ultrasound image is generated.

The image post-processing unit 348 may perform enhancement of the generated ultrasound image automatically or manually by a medical professional or technician to reconstruct the generated image. The reconstructed image is then displayed on a screen of the display 349.

The GPU 340 can process RF frames, from the FIFO buffer 350, at a different rate than the rate that the CPU 330 is receiving the RF data. Thus, by selecting an optimal number of the memory blocks of the FIFO buffer 350, the total process time can be reduced.

L_FIFO is defined as a number of memory blocks 352, 354, 356, 358, etc. of the FIFO buffer 350 and may be greater than (t2+t3)/t1, where t1 is the time for the CPU 330 receiving one RF frame from data transmission and acquisition unit 310, t2 is the time for the GPU 340 performing beamforming of the RF frame, and t3 is the time for the GPU 340 performing compounding, post processing, and display of the reconstructed image.

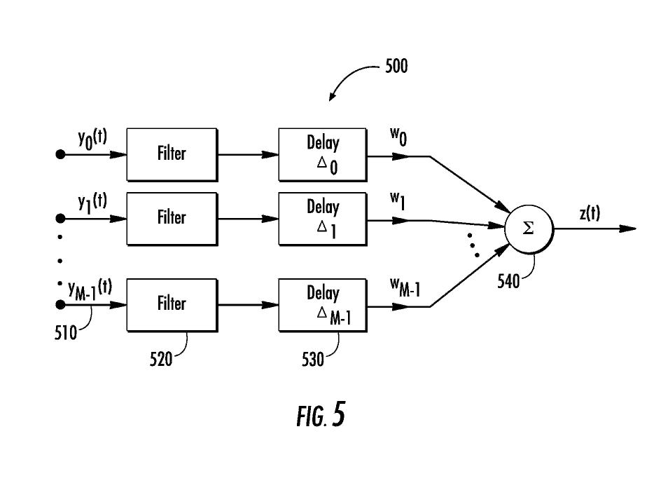

FIGS. 4A and 4B illustrate CPU and GPU working flow 400, where FIG. 4A is the working flow when L_FIFO<(t2+t3)/t1, and FIG. 4B is the working flow when L_FIFO>(t2+t3)/t1, in accordance with aspects of the present disclosure.

When L_FIFO<(t2+t3)/t1, the CPU receiving workflow is represented as 410 and the GPU processing workflow is represented as 420, as shown in FIG. 4A. When L_FIFO>(t2+t3)/t1, the CPU receiving workflow is represented as 430 and the GPU processing workflow is represented as 440, as shown in FIG. 4B.

In one embodiment of the present disclosure, t1 is around 3.33 milliseconds (ms), t2 is around 2 ms, t3 is around 6 ms, and N is 11, where N is a number of different steering angles of the ultrasonic plane waves. With these values, (t2+t3)/t1=(2+6)/3.33 2.42.

Referring back to FIG. 4A, when L_FIFO is less than (t2+t3)/t1, for example L_FIFO is 2, the CPU can store up to two RF frames in the FIFO buffer. Assuming that the CPU starts storing the current first RF frame for the time 412, which is t1, after the GPU has completed reading the previous second last RF frame (in this example, the previous (N−1)-th RF frame), the current second RF frame can be stored in the second memory block for the time 414, which is also t1, after the current first RF frame is stored because the previous N-th RF frame 412 has been read before completion of storing the current first RF frame. After completion of reading the previous N-th RF frame for the time 421, which is t2, the GPU performs the image post-processing for the time 422 (i.e. t3). The GPU completes its reception of the previous N-th RF frame and the image post-processing for 8 ms, which is the sum of the times 421 and 422 or t2 and t3, while the CPU completes reception and storing of the current first and second RF frames for the sum of the times 412 and 414 or 6.66 ms. Thus, the CPU cannot store the current third RF frame for the time 416, 1.67 ms because the GPU can read the current first RF frame only after the sum of the times 421 and 422 or 8 ms. In other words, the time 416, 1.67 ms, is wasted at the CPU.

After storing the current first and second RF frames in the memory blocks, the CPU can store (k−1)-th and k-th RF frames without wasting times because the GPU can read a RF frame faster than the CPU stores a RF frame. Thus, the total time for storing N RF frames by the CPU is t2+t3+(N-L_FIFO)*t1. For example, when N is 11, the total time is 37.97 ms. The total time for processing N RF frames by the GPU is 3*t2+(N−4)*t1+t3, which is 35.31, which is smaller than the total processing time by the CPU.

Referring back to FIG. 4B, when L_FIFO is greater than or equal to (t2+t3)/t1, for example L_FIFO is 3, the CPU can store up to three RF frames of RF data in the FIFO buffer. Assuming that the CPU starts storing the current first RF frame for the time 412, which is t1, after the GPU completes reading the previous second last RF frame (in this example, the previous (N−1)-th RF frame), the current second and third RF frames can be stored in the second and third memory blocks for the times 414 and 416, after the current first RF frame is stored because the previous N-th RF frame 412 has been read before completion of storing the current first RF frame. After completion of reading the previous N-th RF frame for the time 421, the GPU performs the image post-processing for the time 422. The GPU completes its reception of the previous N-th RF frame and the image post-processing for 8 ms. Since there are three memory blocks, the CPU completes reception and storing of the current first to third RF frames for the sum of the times 412, 416, and 418 or 9.99 ms. Since the GPU can read the first RF frame after the 8 ms, the CPU can continuously store the current fourth RF frame and the following RF frames without wasting times. Thus, under this example, the total time processed at the CPU is N*t1. If N is 11, the total time is 36.63 ms, which is smaller than the total time calculated for the case in FIG. 4A.

Therefore, in FIG. 4B, parallelization of the CPU receiving and GPU processing by utilizing an appropriate number of memory blocks of the FIFO buffer can reduce the total processing time at the CPU. Because t1*N>t2*N+t3, the total time is equal to the total time at the CPU. Consequently, with reference to FIGS. 3 and 4B, the minimization of the total time for the processing between the CPU and GPU can be achieved by using an appropriate number of memory blocks of the FIFO buffer based on the reception and processing times of one RF frame by the CPU and GPU, respectively. Thus, the optimized number of memory blocks of the FIFO buffer allows for a faster frame rate. Having an appropriate number of memory blocks, the FIFO buffer is continuously available for receiving RF frames at the CPU, and the GPU reads and processes RF data without delaying the total processing time for one RF frame.

In an aspect, if L_FIFO is set as the minimum value that is greater than or equal to (t2+t3)/t1, the memory can be used efficiently without wasting memory space that is not needed. In this way, each of the plurality of memory blocks of the FIFO buffer is optimally utilized based on the processing times of one RF frame by the CPU and the GPU.

FIG. 5 illustrates a block diagram 500 showing beamforming with temporal delays in accordance with aspects of the present disclosure. As described above with respect to the transducer elements of the transducer 110 of FIG. 1, the transducer elements use temporal delays while generating ultrasound waveforms. Likewise, the beamforming uses temporal delays. For example, if the first transducer generates an ultrasound waveform by delaying a temporal period, the beamforming also delays the same temporal period for the RF data corresponding to the ultrasound waveform generated by the first transducer element.

For example, FIG. 5 shows M scan lines meaning that the RF frame is generated by M transducer elements. For example, input RF data 510, yₖ(t), may be obtained from the (k+1)-th transducer element, is filtered by a filter 520, and temporarily delayed by a period Δₖ, which corresponds the temporal delay for the (k+1)-th transducer element, where k=0, 1, 2, . . . , M−1. All delayed RF data 635, wₖ, are then summed to output an output data 550, z(t), which is a pixel value for a pixel in the ultrasound image.

In order to reduce the time for calculating corresponding positions in RF data from the plurality of transducer elements, N_Steer mapping tables are calculated before beamforming, and stored in the mapping tables in a 2-D texture memory. N_Steer is the number of steering angles when the ultrasound probe transmits plane waves, and the size of every mapping table is N*W_RF, where W_RF is the number of lines in the RF data and N is the number of pixels in a reconstructed image. Thus, every pixel in the reconstructed image is generated from and calculated by adding W_RF points of RF data together via the summer 540.

In one embodiment, the transmit circuitry may be configured to operate the transducer array 110 such that the acoustic energy emitted is directed or steered as plane waves. For example, a processing circuitry may impart respective time delays 530 (FIG. 5) to generate temporally offset pulsed waveforms that are applied to respective transducer elements. These temporal offsets result in different activation times of the respective transducer elements such that the waveform of acoustic energy emitted by the transducer array 110 is effectively steered or directed in a particular direction with respect to the surface of the transducer array 110.

Thus, by adjusting the time delays 530 associated with the pulsed waveforms that energize the respective transducer elements, the ultrasonic plane waves can be directed toward or away from an axis associated with the surface of the transducer array 110 by a specified angle (0) and focused at a fixed range within the patient tissue. In such an implementation, a sector scan may be performed by progressively changing the time delays in successive excitations. The steering angle θ is thus incrementally changed to steer the transmitted plane wave in a succession of steering directions.

The echo signals produced by each burst of acoustic energy are reflected by structures or structure interfaces or target tissue located at successive ranges along the ultrasonic plane waves. The echo signals are sensed separately by each transducer element and a sample of the echo signal magnitude at a particular point in time represents the amount of reflection occurring at a specific range.

The beamformer 342 may be implemented by a programmable logic device. The programmable logic device filters, interpolates, demodulates, phases, applies apodization, delays and/or sums the received signals, which are functions of the beamformer 342. The programmable logic device digitally controls the delays and characteristic of transmit waveforms, and generates transmit waveforms from memory, which are functions of the transmit waveform. The programmable logic device may also implement relative delays between the waveforms as well as filter, interpolate, modulate, phase, and apply apodization. The programmable logic device controlling the beamformer 342 to perform functions to process the plurality of signals associated with such multi-element electrically scanned arrays.

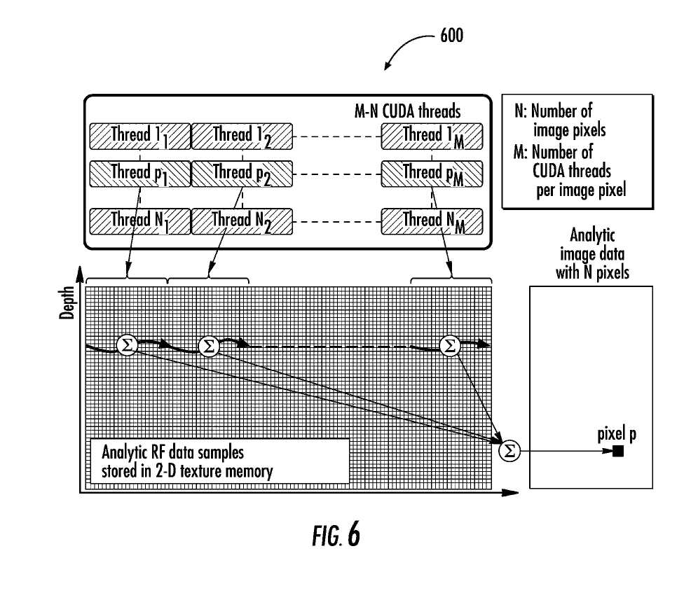

FIG. 6 illustrates data-level parallelism 600 in delay-and-sum operations, in accordance with aspects of the present disclosure.

To reconstruct an image with N pixels, M−N CUDA threads are created and M threads are assigned to each pixel, where M is empirically optimized for each imaging application. The threads assigned to adjacent pixels are grouped together in the same thread block to maximize the memory access efficiency by utilizing the spatial locality of the raw data samples stored in the 2-D texture memory. CUDA® is a parallel computing platform and programming model invented by NVIDIA®. It enables dramatic increases in computing performance by harnessing the power of the GPU.

In prior approaches, a good amount of time was wasted to populate delay tables and to find points in the tables that correspond to a pixel in the reconstructed image. In the present disclosure, before performing the beamforming, the delay table is populated and stored in the GPU so that the GPU can perform a look-up in the table to find relevant positions in the 3-D texture memory and add them together, as shown in FIG. 7. This GPU reconstruction approach is faster than prior approaches because performing look-ups in the pre-stored table is faster than populating/repopulating the delay table before each beamforming. In one embodiment, the ultrasound system of the present disclosure only needs to populate N_Steer delay tables (one per each steering angle) and store them in FIFO buffers. As a result, when performing beamforming, embodiments of the present disclosure only need to perform look-ups in the table and do not need to repopulate the table.

Advantages of the present disclosure further include adding FIFO buffers to make sure the RF data received by the CPU and image reconstruction with the GPU are performed in parallel. The CPU continuously receives and stores digital RF data in memory blocks of the FIFO buffer when the FIFO buffer is not full. The GPU reads the RF data from the memory blocks of the FIFO buffer when the memory blocks are not empty, and performs beamforming, compounding, post processing, and display. The size of one memory block of the FIFO buffer is the same as the size of one RF frame. Therefore, each RF frame is stored in one memory block of the FIFO buffer and the GPU only needs to read one memory block of the FIFO buffer to get a RF frame, and then performs the beamforming processing. In order to reduce the whole processing time, L_FIFO may be greater than or equal to (t2+t3)/t1.

There are many transducer array systems contemplated by the disclosed embodiments. Most of the description focuses on a description of a diagnostic medical ultrasound system, however, the disclosed embodiments are not so limited. The description focuses on diagnostic medical ultrasound systems solely for the purposes of clarity and brevity. It should be appreciated that disclosed embodiments apply to numerous other types of methods and systems.

In a transducer array system, the transducer array is used to convert a signal from one format to another format. For example, with ultrasound imaging the transducer converts an ultrasonic wave into an electrical signal, while a radar system converts an electromagnetic wave into an electrical signal. While the disclosed embodiments are described with reference to an ultrasound system, it should be appreciated that the embodiments contemplate application to many other systems. Such systems include, without limitation, radar systems, optical systems, and audible sound reception systems.

Additionally, “code” as used herein, or “program” as used herein, may be any plurality of binary values or any executable, interpreted or compiled code which may be used by a computer or execution device to perform a task. This code or program may be written in any one of several known computer languages. A “computer,” as used herein, may mean any device which stores, processes, routes, manipulates, or performs like operation on data. A “computer” may be incorporated within one or more ultrasound imaging devices or one or more electronic devices or servers to operate one or more processors to run the ultrasound imaging devices. It is to be understood, therefore, that this disclosure is not limited to the particular forms illustrated and that it is intended in the appended claims to embrace all alternatives, modifications, and variations which do not depart from the spirit and scope of the embodiments described herein.

Detailed embodiments of devices, systems incorporating such devices, and methods using the same as described herein. However, these detailed embodiments are merely examples of the disclosure, which may be embodied in various forms. Therefore, specific structural and functional details disclosed herein are not to be interpreted as limiting, but merely as a basis for the claims and as a representative basis for allowing one skilled in the art to variously employ the present disclosure in appropriately detailed structure.

As will be appreciated, as used herein the term “circuitry” may describe hardware, software, firmware, or some combination of these which are configured or designed to provide the described functionality, such as transmit beamforming, receive beamforming, and/or scan conversion.

The term “delay” is intended broadly to encompass both delaying and advancing one signal relative to another.

The term “module” may at least refer to a self-contained component (unit or item) that is used in combination with other components and/or a separate and distinct unit of hardware or software that may be used as a component in a system, such as an ultrasound system including a transducer array having a plurality of transducer elements. The term “module” may also at least refer to a self-contained assembly of electronic components and circuitry, such as a stage in a computer that is installed as a unit. The term “module” may be used interchangeably with the term “unit.”

The term “storage” may refer to at least data storage. “Data storage” may at least refer to any article or material (e.g., a hard disk) from which information is capable of being reproduced, with or without the aid of any other article or device. “Data storage” may at least refer to the holding of data in an electromagnetic form for access by a computer processor. Primary storage is data in random access memory (RAM) and other “built-in” devices. Secondary storage is data on hard disk, tapes, and other external devices. “Data storage” may also at least refer to the permanent holding place for digital data, until purposely erased. “Storage” implies a repository that retains its content without power. “Storage” mostly means magnetic disks, magnetic tapes and optical discs (CD, DVD, etc.). “Storage” may also refer to non-volatile memory chips such as flash, Read-Only memory (ROM) and/or Electrically Erasable Programmable Read-Only Memory (EEPROM).

The term “processing” may at least refer to determining the elements or essential features or functions or processes of one or more ultrasound imaging devices for computational processing. The term “process” may further refer to tracking data and/or collecting data and/or manipulating data and/or examining data and/or updating data on a real-time basis in an automatic manner and/or a selective manner and/or manual manner (continuously, periodically or intermittently).

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. An ultrasound imaging system comprising:
a transducer array including a plurality of transducer elements, each of the plurality of transducer elements configured to transmit acoustic energy to a region of interest and receive reflected acoustic energy;
an ultrasound frontend configured to digitally sample the reflected acoustic energy to generate radio frequency (RF) data; and
a processing apparatus including:
a central processing unit (CPU) for receiving the RF data including RF frames;
a first in/first out (FIFO) buffer including a plurality of memory blocks for storing RF frames, wherein a size of each memory block is equal to the size of a single RF frame; and
a graphics processing unit (GPU) for reading the RF frames from the plurality of memory blocks of the FIFO buffer and reconstructing an image.

2. The ultrasound imaging system according to claim 1, further comprising a display for displaying the reconstructed image of the region of interest.

3. The ultrasound imaging system according to claim 1, wherein the image is reconstructed by performing envelope detection, compounding, and post-processing.

4. The ultrasound imaging system according to claim 1, wherein a number of the plurality of memory blocks of the FIFO buffer is greater than or equal to (t2+t3)/t1, where t1 is the time that the CPU receives one RF frame, t2 is the time that the GPU reads one RF frame, and t3 is the time that the GPU performs envelope detection, compounding, and post-processing.

5. The ultrasound imaging system according to claim 1, wherein the CPU receives the RF frames and the GPU reads the RF frames, in a parallel manner.

6. The ultrasound imaging system according to claim 1, wherein the number of the plurality of transducer elements is 128.

7. The ultrasound imaging system according to claim 1, wherein the acoustic energy is transmitted in plane waveform.

8. The ultrasound imaging system according to claim 7, wherein the plane waveform has 11 steering angles.

9. The ultrasound imaging system according to claim 1, wherein the GPU reads a single memory block of the FIFO buffer to process one RF frame.

10. The ultrasound imaging system according to claim 1, wherein the GPU performs beamforming processing by delay-and-sum operations in a parallel manner.

11. An ultrasound imaging method comprising:
transmitting acoustic energy to a region of interest by a transducer array including a plurality of transducer elements;
receiving reflected acoustic energy;
digitally sampling the reflected acoustic energy to generate RF data;
receiving the RF data including RF frames by a central processing unit (CPU);
storing a RF frame in a memory block of a plurality of memory blocks of a first in/first out (FIFO) buffer, where a size of each memory block is equal to the size of a single RF frame;
reading the RF frame by a graphics processing unit (GPU) from the memory block of the FIFO buffer; and
reconstructing an image based on the RF frame by the GPU.

12. The method according to claim 11, further comprising displaying the reconstructed image of the region of interest on a display.

13. The method according to claim 11, wherein reconstructing the image includes performing envelope detection, compounding, and post-processing by the GPU.

14. The method according to claim 11, wherein a size of each memory block of the FIFO buffer is greater than or equal to (t2+t3)/t1, where t1 is the time that the CPU receives one RF frame, t2 is the time that the GPU reads the RF frame from the memory block, and t3 is the time that the GPU reconstructs the image.

15. The method according to claim 11, wherein receiving the RF data and reading the RF frame are performed in a parallel manner.

16. The method according to claim 11, wherein the number of the plurality of transducer elements is 128.

17. The method according to claim 16, wherein the acoustic energy is transmitted in plane waveform.

18. The method according to claim 17, wherein of the plane waveform has 11 steering angles.

19. The method according to claim 17, wherein the beamforming processing is performed by delay-and-sum operations in a parallel manner.

20. The method according to claim 11, further comprising performing beamforming process on the RF frame by the GPU.
